(19) United States
(12) Patent Application Publication
(10) Pub. No.: US 2025/0268327 A1
(43) Pub. Date: Aug. 28, 2025

(54) Bonneye

(71) Applicant: Althea Genae Collins, Hephzibah, GA (US)

(72) Inventor: Althea Genae Collins, Hephzibah, GA (US)

(73) Assignee: Althea Genae Collins, Hephzibah, GA (US)

(21) Appl. No.: 18/590,818

(22) Filed: Feb. 28, 2024

(51) Int. Cl. A42B 1/049; A61F 9/04

(52) U.S. Cl. CPC A42B 1/049; A61F 9/04

(57) ABSTRACT

The nature of this invention is to combine what represents as a bonnet for hair protection alongside an eye mask for sleep, mediation, and relaxation. A bonnet will be molded to an eye mask. The construction form includes detachable, retractable, and convertible eye mask. This invention creates a space for deep sleep knowing one's hair, edges, eyes, and eyelashes are protected. The invention protects the hair from dust, lint, debris, and friction against fabric that may lead to breakage, dry and damaged hair. In addition to being connected to an eye mask, protecting the eyes from light and brain distractions, improving the quality of sleep. The connection between the bonnet and the eye mask will be used as a strap and band.

### 2. CROSS-REFERENCE TO RELATED APPLICATIONS

a. Not applicable

### 3. STATEMENT REGARDING FEDERALLY SPONSORED RESEARCH DEVELOPMENT

a. Not applicable

### 4. REFERENCE TO A “SEQUENCE LISTING” (IF APPLICABLE)

a. Not applicable

### 5. BACKGROUND OF THE INVENTION

a. One field that the advanced invention ‘Bonneye’ falls in is the beauty industry. A second field that Bonneye falls into is the therapy of self-care and well-being. The problem with just a bonnet is that it only protects the hair, but when combined with an eye mask it protects the hair, eyes, and promotes calmness and relaxation. The Bonneye is placed on the head and over the hair of a body and the covering/mask component over the eyes. The affixed structure includes easier access to both dependent items. Prior, the bonnet may have been placed under the eye mask or over the eye mask. With this invention the two are placed together, reducing loss of one item or misplacing the other item. The Bonneye uses multiple attachment techniques: glue, button, staples, clips, snaps and plastic insert buttons, Velcro, elastic, magnet, hooks, twist, draw strings, fasteners, zipper, and materials attached by sewing method. The Bonneye increases control mentally of the mindset of comfort, ease of access, protection, and beauty. This therapeutic invention helps one to reduce unwanted distractions and dirt. Also, representing self-care increasing well-being and happiness when one wakes up happier with nice intact hair and non-restless eyes.

### 6. DESCRIPTION OF PRIOR ARTS

a. U.S. Pat. No. 7,895,672B2 the functionality of the prior art product relates to a night cap which comprises a cap body, intended for hair protection of the handler.

b. U.S. Pat. No. 4,872,217A The prior art invention relates to an eye mask, principally for improvement allowing good sleep such that the mask fits at ease on a face without clearly contacting an eyeball of a handler.

### 7. BRIEF SUMMARY OF THE INVENTION

a. A design to attach a mask and bonnet as one unit or component. Separately worn are hair pieces to protect the hair growth, follicles, and strands. Eye mask helps promote sleep, decrease distractions, and enhance meditation and relaxation. The invention of combining two objects to make a meaningful unit mention for better sleep, better hair, and better days. Those individuals that are interested in hair protection whether it is day or night will be drawn to the Bonneye because it can be used anytime and worn anywhere. Also, those interested in light blocking, eye rest, and less distractions will benefit from Bonneye as its interchangeable component is attached to protect the eyes. The object of the invention is to avoid hair damage and eye de-sensitivity by attaching two dependent items to satisfy one unique pattern for sleep protection.

### 8. BRIEF DESCRIPTION OF DRAWINGS

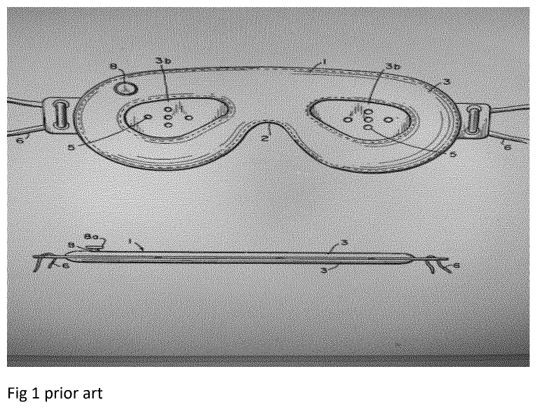

a. FIG. 1 shows a front view of an eye mask of a conventional type.

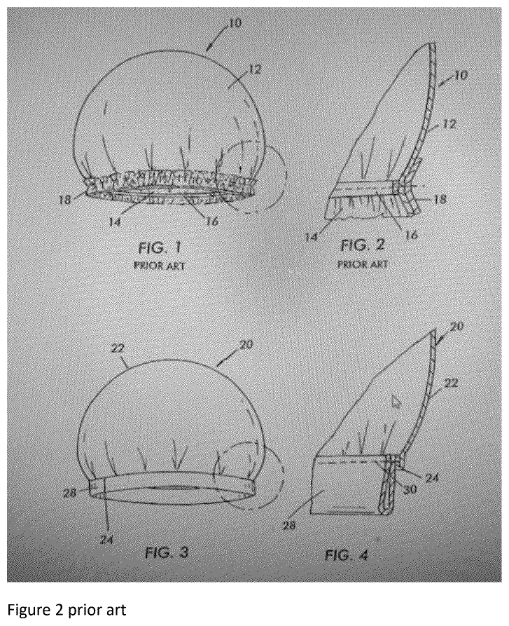

b. FIG. 2 illustrates a sleep bonnet according to the prior art.

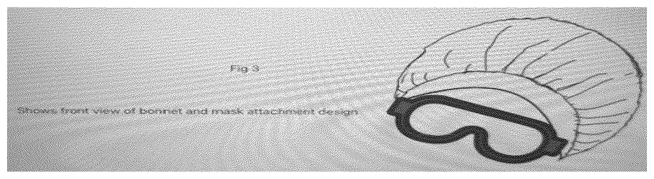

c. FIG. 3 illustrates a sleep bonnet attached to a mask, according to the invention (front view), main member.

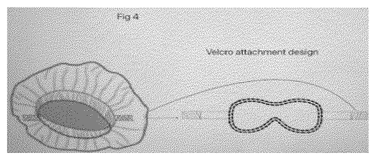

d. FIG. 4 shows Velcro attachment design (front view).

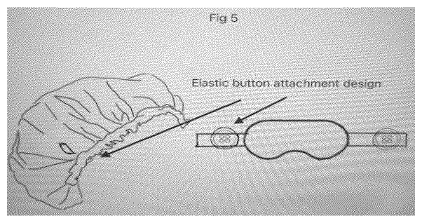

e. FIG. 5 shows elastic button attachment design (side view).

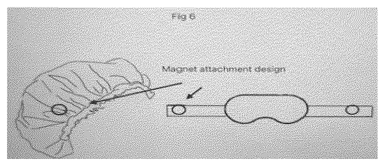

f. FIG. 6 shows magnet attachment design (side view).

g. FIG. 7 shows hook attachment design (front view).

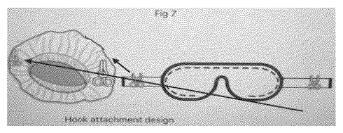

h. FIG. 8 shows glued attachment design (side view).

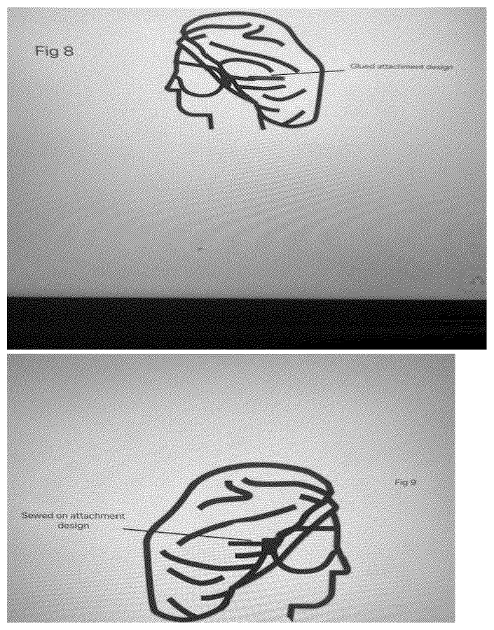

i. FIG. 9 shows sewed attachment design (side view).

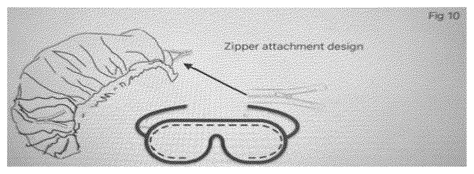

j. FIG. 10 shows zipper attachment design (side view).

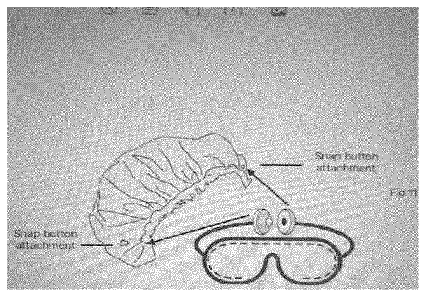

k. FIG. 11 shows snap button attachment design (side view).

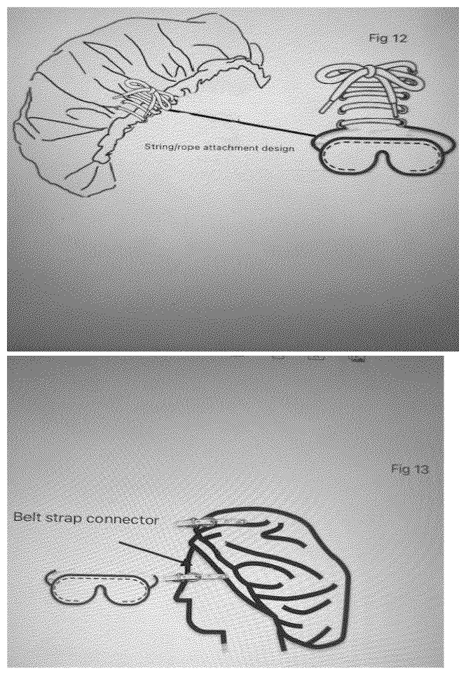

l. FIG. 12 shows string/rope attachment design (side view).

m. FIG. 13 shows belt strap connector design (side view).

### 9. DETAILED DESCRIPTION OF THE DRAWINGS EMBODIMENT

a. FIG. 1 illustrates a conventional type of eye mask, placement on the human face for sleeping purposes. FIG. 2 illustrates prior art sleeping bonnet, flexible material hair covering.

b. FIG. 3 illustrates the main member attachment design of the sleep bonnet and sleep mask, so that said member is forming as one component and unit, according to the invention.

c. FIG. 4 shows the main member method attached by Velcro, according to the invention. FIG. 5 shows the main member method attached by elastic buttons, according to the invention. FIG. 6 shows the main member method attachment design by magnets, according to the invention. FIG. 7 illustrates the main member method attachment design by hooks, according to the invention. FIG. 8 shows the main member method stationary design by glued attachment, according to the invention. FIG. 9 illustrates the main member method stationary sewn design, according to the invention. FIG. 10 shows the main member method attached by zippers, according to the invention. FIG. 11 shows a design attachment method by snap button assemblies on the main member, according to the invention. FIG. 12 shows the main member design attachment method by string/rope, according to the invention. FIG. 13 the main member being attached by strap, according to the invention.

### 10. DETAILED DESCRIPTION OF THE INVENTION

a. The present invention relates to a hair and eye protective combination design comprising of satin, elastic, plastic, polyester, cotton, fleece, and cloth material that can be fabricated as a unique look on a one-piece unit. A hair bonnet also known as hair cap—a cap of woolen fabric affixed to an eye mask made of silk or satin, soft luxurious material with light blocking capability-covering used for sleep.

b. The method of the two using three exchangeable methods: attachable via velcro, buttons, and sewing thread.

c. Amendable via the ability to adjust mask to use over eyes or place above bonnet and only use bonnet.

d. Convertible by the flexibility to use bonnet and detach mask; use mask and detach bonnet.

e. The Bonneye's intended use is to combine a hair bonnet with an eye mask. The Bonneye can be worn for sleep on an airplane, in the bed, daytime on the sofa, in the car on the way to a hair appointment, in the shower, and anywhere one sees fits the need to protect the eyes and hair from exposure.

f. The bonneye protects the hair and eyes from light, debris, particles, weather conditions, brain distractions, and damaged hair.

g. The Bonneye separates itself from previous products because it promotes convenience and accessibility of having both items together vs having to use each independently. The purpose of the Bonneye is to promote rest, relaxation, hair and eye protection.

What I claim is:

1. A bonnet and eye mask designed for wearing on the head and over the eyes that is comprising the combination of connected and made whole pieces and is considered one unit and component. (Independent claim).

2. The bonnet and eye mask unit of claim 1 is made of fabric and plastic. (further limiting).

3. The bonnet and eye mask of claim 1 is attached using buttons, staples, clips, hooks, fasteners and zippers (additional structures).

4. The bonnet and eye mask of claim 1 is connected using Velcro, elastic, twisting, and draw strings (additional structures).

5. Bonnet and eye mask of claim 1 is fixed using glue, magnets, and sewing articles. (additional structures).
